(19) United States
(12) Patent Application Publication
(10) Pub. No.: US 2016/0136587 A1
(43) Pub. Date: May 19, 2016

(54) ZWITTERION-FUNCTIONALIZED COPOLYMER HOLLOW-FIBER MEMBRANES AND ASSOCIATED METHOD

(71) Applicant: General Electric Company, Schenectady, NY (US)

(72) Inventors: Patrick Joseph McCloskey, Watervliet, NY (US); Cathryn Ellen Olsen, Wilton, NY (US); Hongyi Zhou, Niskayuna, NY (US); Louisa Ruth Carr, Glenville, NY (US); Matthew Jeremiah Misner, Delanson, NY (US)

(21) Appl. No.: 14/547,306

(22) Filed: Nov. 19, 2014

(51) Int. Cl. B01D 71/68; B01D 67/00; B01D 69/02; B01D 69/08; A61M 1/16

(52) U.S. Cl. CPC B01D 71/68; B01D 69/08; A61M 1/16; B01D 69/02; B01D 67/0002; B01D 2325/42; B01D 2323/00

(57) ABSTRACT

A hollow-fiber membrane is presented. The hollow fiber membrane, includes a copolymer including sulfone structural units having a formula (I) and zwitterion-functionalized structural units having a formula (II). Method of making the hollow-fiber membrane is also presented.

### BACKGROUND

The invention generally relates to zwitterion-functionalized copolymer membranes and methods of making these. More particularly, the invention relates to zwitterion-functionalized copolymer hollow-fiber membranes and methods of making these.

Porous hollow-fiber polymeric membranes may be employed in many applications, such as, hemodialysis, ultrafiltration, nanofiltration, reverse osmosis, gas separation, microfiltration, and pervaporation. For many of these applications, membranes with optimal selectivity as well as chemical, thermal and mechanical stability are desirable. In many applications (for example, bio-separation or water filtration) it may also be desirable to have membranes with one or more of improved hydrophilicity, improved biocompatibility, or low fouling.

Polyarylene ethers, in particular, polyethersulfones and polysulfones are often used as membrane materials because of their mechanical, thermal, and chemical stability. However, these polymers may not have the optimal biocompatibility and hydrophilicity for many applications. Further improvements in membrane hydrophilicity have been achieved by polymer blending, for example, fabricating the porous membrane in the presence of small amounts of hydrophilic polymers such as polyvinylpyrollidone (PVP). However, since PVP is water-soluble it is slowly leached from the porous polymer matrix creating product variability. Alternatively, hydrophilicity has been achieved via functionalization of the polymer backbone and introduction of carboxyl, nitrile or polyethylene glycol functionality. However, these chemical modifications may be complicated, expensive and inefficient. Further, addition of the functional groups may make it difficult to fabricate hollow-fiber membranes from the functionalized polymers.

Thus, hollow-fiber membranes having one or both of optimal hydrophilicity and biocompatibility are desired. Further, methods of fabricating these hollow-fiber membranes are also desired.

### BRIEF DESCRIPTION OF THE INVENTION

Embodiments of the present invention are included to meet these and other needs. One embodiment is a hollow-fiber membrane, including a copolymer comprising sulfone structural units having a formula (I) and zwitterion-functionalized structural units having a formula (II):

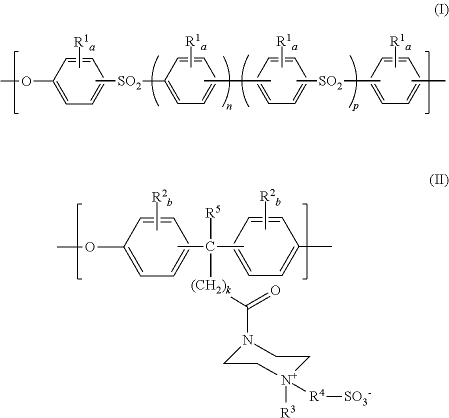

wherein “a” and “b” are independently at each occurrence 0, 1, 2, 3, or 4;
“n” and “p” are independently 0 or 1;
“k” is in a range from 0 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical;
R³ and R⁵ are independently a hydrogen atom, a C₁-C₁₂ aliphatic radical, a C₃-C₁₂ cycloaliphatic radical, or a C₃-C₁₂ aromatic radical;
R⁴ is a bond, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

One embodiment is a hollow-fiber membrane, including a copolymer comprising sulfone structural units having a formula (III) and zwitterion-functionalized structural units having a formula (IV):

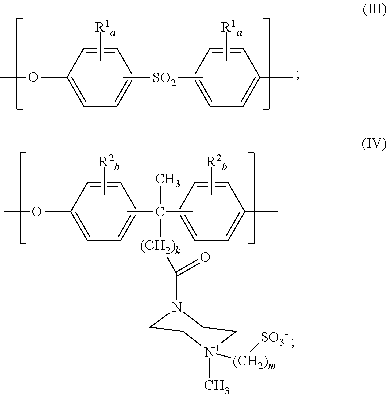

wherein “a” and “b” are independently at each occurrence 0, 1, 2, 3, or 4;
“k” is a number in a range from 0 to 10;
“m” is a number in a range from 1 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

One embodiment is a method of forming a hollow-fiber membrane. The method includes: (A) providing a casting solution comprising a copolymer and a solvent, wherein the copolymer comprises sulfone structural units having a formula (I) and zwitterion-functionalized structural units having a formula (II):

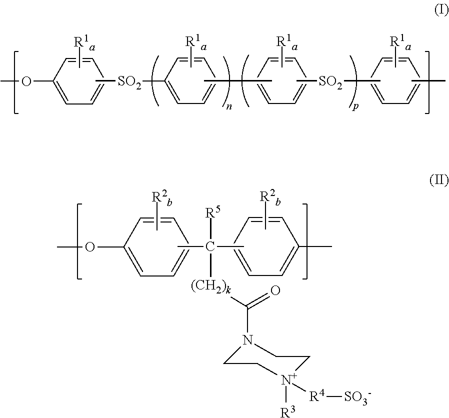

wherein “a” and “b” are independently at each occurrence 0, 1, 2, 3, or 4;
“n” and “p” are independently 0 or 1;
“k” is in a range from 0 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical;
R³ and R⁵ are independently a hydrogen atom, a C₁-C₁₂ aliphatic radical, a C₃-C₁₂ cycloaliphatic radical, or a C₃-C₁₂ aromatic radical;
R⁴ is a bond, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent. The method further includes (B) extruding the casting solution through an annular channel to form the hollow-fiber membrane.

### DETAILED DESCRIPTION

Approximating language, as used herein throughout the specification and claims, may be applied to modify any quantitative representation that could permissibly vary without resulting in a change in the basic function to which it is related. Accordingly, a value modified by a term or terms, such as “about”, and “substantially” is not to be limited to the precise value specified. In some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

In the following specification and the claims, the singular forms “a”, “an” and “the” include plural referents unless the context clearly dictates otherwise. As used herein, the term “or” is not meant to be exclusive and refers to at least one of the referenced components being present and includes instances in which a combination of the referenced components may be present, unless the context clearly dictates otherwise.

As used herein, the term “aromatic radical” refers to an array of atoms having a valence of at least one comprising at least one aromatic group. The array of atoms having a valence of at least one comprising at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical, which comprises a phenyl ring (the aromatic group) and a methylene group (the nonaromatic component). Similarly a tetrahydronaphthyl radical is an aromatic radical comprising an aromatic group (C₆H₃) fused to a nonaromatic component —(CH₂)₄₋. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical comprising a methyl group, the methyl group being a functional group which is an alkyl group. Similarly, the 2-nitrophenyl group is a C₆ aromatic radical comprising a nitro group, the nitro group being a functional group. Aromatic radicals include halogenated aromatic radicals such as 4-trifluoromethylphenyl, hexafluoroisopropylidenebis(4-phen-1-yloxy) (i.e., —OPhC(CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-), and the like. Further examples of aromatic radicals include 4-allyloxyphen-1-oxy, 4-aminophen-1-yl (i.e., 4-H₂NPh-), 3-aminocarbonylphen-1-yl (i.e., NH₂COPh-), 4-benzoylphen-1-yl, dicyanomethylidenebis(4-phen-1-yloxy) (i.e., —OPhC(CN)₂PhO—), 3-methylphen-1-yl, methylenebis(4-phen-1-yloxy) (i.e., —OPhCH₂PhO—), 2-ethylphen-1-yl, phenylethenyl, 3-formyl-2-thienyl, 2-hexyl-5-furanyl, hexamethylene-1,6-bis(4-phen-1-yloxy) (i.e., —OPh(CH₂)₆PhO—), 4-hydroxymethylphen-1-yl (i.e., 4-HOCH₂Ph-), 4-mercaptomethylphen-1-yl (i.e., 4-HSCH₂Ph-), 4-methylthiophen-1-yl (i.e., 4-CH₃SPh-), 3-methoxyphen-1-yl, 2-methoxycarbonylphen-1-yloxy (e.g., methyl salicyl), 2-nitromethylphen-1-yl (i.e., 2-NO₂CH₂Ph), 3-trimethylsilylphen-1-yl, 4-t-butyldimethylsilylphenl-1-yl, 4-vinylphen-1-yl, vinylidenebis(phenyl), and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein the term “cycloaliphatic radical” refers to a radical having a valence of at least one, and comprising an array of atoms which is cyclic but which is not aromatic. As defined herein a “cycloaliphatic radical” does not contain an aromatic group. A “cycloaliphatic radical” may comprise one or more noncyclic components. For example, a cyclohexylmethyl group (C₆H₁₁CH₂—) is a cycloaliphatic radical which comprises a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylcyclopent-1-yl radical is a C₆ cycloaliphatic radical comprising a methyl group, the methyl group being a functional group which is an alkyl group. Similarly, the 2-nitrocyclobut-1-yl radical is a C₄ cycloaliphatic radical comprising a nitro group, the nitro group being a functional group. A cycloaliphatic radical may comprise one or more halogen atoms, which may be the same or different. Halogen atoms include, for example; fluorine, chlorine, bromine, and iodine. Cycloaliphatic radicals comprising one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂C₆H₁₀—), 2-chloromethylcyclohex-1-yl, 3-difluoromethylenecyclohex-1-yl, 4-trichloromethylcyclohex-1-yloxy, 4-bromodichloromethylcyclohex-1-ylthio, 2-bromoethylcyclopent-1-yl, 2-bromopropylcyclohex-1-yloxy (e.g., CH₃CHBrCH₂C₆H₁₀O—), and the like. Further examples of cycloaliphatic radicals include 4-allyloxycyclohex-1-yl, 4-aminocyclohex-1-yl (i.e., H₂NC₆H₁₀—), 4-aminocarbonylcyclopent-1-yl (i.e., NH₂COC₅H₈—), 4-acetyloxycyclohex-1-yl, 2,2-dicyanoisopropylidenebis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, cyclopropylethenyl, 3-formyl-2-terahydrofuranyl, 2-hexyl-5-tetrahydrofuranyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃S C₆H₁₀—), 4-methoxycyclohex-1-yl, 2-methoxycarbonylcyclohex-1-yloxy (2-CH₃OCOC₆H₁₀O—), 4-nitromethylcyclohex-1-yl (i.e., NO₂CH₂C₆H₁₀—), 3-trimethylsilylcyclohex-1-yl, 2-t-butyldimethylsilylcyclopent-1-yl, 4-trimethoxysilylethylcyclohex-1-yl (e.g., (CH₃O)₃SiCH₂CH₂C₆H₁₀—), 4-vinylcyclohexen-1-yl, vinylidenebis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein the term “aliphatic radical” refers to an organic radical having a valence of at least one consisting of a linear or branched array of atoms which is not cyclic. Aliphatic radicals are defined to comprise at least one carbon atom. The array of atoms comprising the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical comprising a methyl group, the methyl group being a functional group which is an alkyl group. Similarly, the 4-nitrobut-1-yl group is a C₄ aliphatic radical comprising a nitro group, the nitro group being a functional group. An aliphatic radical may be a haloalkyl group which comprises one or more halogen atoms which may be the same or different. Halogen atoms include, for example; fluorine, chlorine, bromine, and iodine. Aliphatic radicals comprising one or more halogen atoms include the alkyl halides trifluoromethyl, bromodifluoromethyl, chlorodifluoromethyl, hexafluoroisopropylidene, chloromethyl, difluorovinylidene, trichloromethyl, bromodichloromethyl, bromoethyl, 2-bromotrimethylene (e.g., —CH₂CHBrCH₂—), and the like. Further examples of aliphatic radicals include allyl, aminocarbonyl (i.e., —CONH₂), carbonyl, 2,2-dicyanoisopropylidene (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), t-butyldimethylsilyl, 3-trimethyoxysilylpropyl (i.e., (CH₃O)₃SiCH₂CH₂CH₂—), vinyl, vinylidene, and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

As discussed in detail below, some embodiments of the invention are directed to a hollow-fiber membrane composed of a copolymer. The copolymer may be a block copolymer or a random copolymer. A block copolymer contains blocks of monomers of the same type that may be arranged sequentially, while a random copolymer contains a random arrangement of the multiple monomers making up the copolymer. In certain embodiments, the copolymer is a random copolymer. Further, the term “copolymer” as used herein refers to the zwitterion-functionalized copolymer, unless the context clearly indicates otherwise.

The term “hollow-fiber membrane” as used herein refers to fiber-based membrane structures including separating layers present at the surface. The hollow-fiber membrane may function using “inside-outside” or “outside-inside” mechanism. The terms “hollow-fiber membrane” and “membrane” are used herein interchangeably, unless the context clearly indicates otherwise.

The copolymer in accordance with some embodiments of the invention includes sulfone structural units having a formula (I) and zwitterion-functionalized structural units having a formula (II):

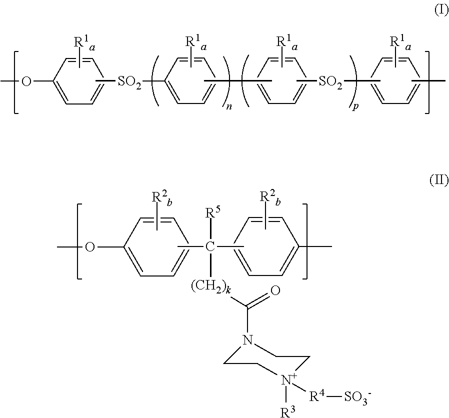

wherein “a” and “b” are independently at each occurrence 0, 1, 2, 3, or 4;
“n” and “p” are independently 0 or 1;
“k” is in a range from 0 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical;
R³ and R⁵ are independently a hydrogen atom, a C₁-C₁₂ aliphatic radical, a C₃-C₁₂ cycloaliphatic radical, or a C₃-C₁₂ aromatic radical;
R⁴ is a bond, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

In certain embodiments, the sulfone structural units have a formula (III):

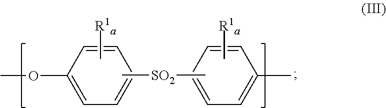

wherein “a” is independently at each occurrence 0, 1, 2, 3, or 4; and
R¹ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

As noted earlier, the copolymer further includes zwitterion functional groups. The term “zwitterion functional group” as used herein refers to a moiety including both positively and negatively charged groups in the same molecule. Without being bound by any theory, it is believed that the zwitterion functional groups may provide improved hydrophilicity and biocompatibility for the copolymer while maintaining membrane-formation capability.

In certain embodiments, the zwitterion-functionalized structural units have a formula (IV):

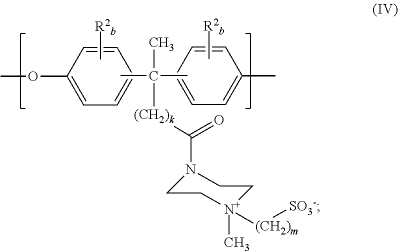

wherein “b” is independently at each occurrence 0, 1, 2, 3, or 4;
“k” is a number in a range from 0 to 10;
“m” is a number in a range from 1 to 10;and
R² is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

The inventors of the present invention have discovered that the amount of zwitterion functional groups in the copolymer has to be maintained in a certain range such that the copolymer may be spun into hollow-fiber membranes while providing the desired protein adhesion performance. For example, copolymers having greater than 50 mole percent of zwitterion functional groups may not be spun effectively to form the hollow-fiber membranes. Copolymers having less than 20 mole percent of zwitterion functional groups may not be provide the desired protein adhesion performance. In some embodiments, the desired protein adhesion performance may be defined as the relative protein adhesion observed for the copolymer versus a commercially available polysulfone (PSU), and having a value less than about 0.5.

In some embodiments, a mole fraction of the zwitterion functional groups in the copolymer is in a range from about 20 mole percent to about 50 mole percent percent. In some embodiments, a mole fraction of the zwitterion functional group in the copolymer is in a range from about 30 mole percent to about 50 mole percent. In some embodiments, a mole fraction of the zwitterion functional group in the copolymer is in a range from about 35 mole percent to about 50 mole percent.

The copolymer may further include structural units having a formula (V):

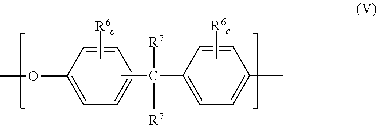

wherein “c” is independently at each occurrence 0, 1, 2, 3, or 4;
R⁶ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
R⁷ is independently at each occurrence a C₁-C₁₂ aliphatic radical, a C₃-C₁₂ cycloaliphatic radical, or a C₃-C₁₂ aromatic radical.

A hollow-fiber membrane, including a copolymer including sulfone structural units having a formula (III) and zwitterion-functionalized structural units having a formula (IV) is also presented:

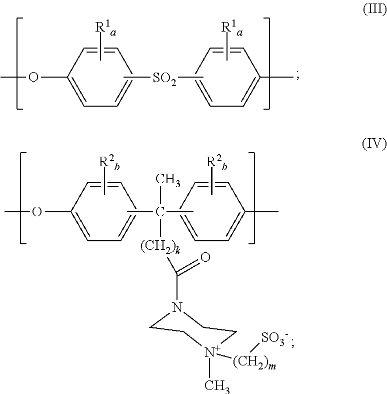

wherein “a” and “b” are independently at each occurrence 0, 1, 2, 3, or 4;
“k” is a number in a range from 0 to 10;
“m” is a number in a range from 1 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

In some such instances, the mole fraction of the zwitterion-functionalized structural units in the copolymer is in a range from about 30 mole percent to about 50 mole percent. The copolymer may further include structural units having a formula (V) in some embodiments.

The copolymers may be synthesized using any suitable techniques. In certain embodiments, the copolymer may be synthesized by reacting at least one aromatic dihydroxy compound with at least one aromatic dihalide compound. At least one of the aromatic dihydroxy compound and the aromatic dihalide compound may be functionalized with a suitable functional group (for example, piperazine amide group) capable of being converted to the zwitterion functional group. In some embodiments, the aromatic dihydroxy compound may be functionalized with the suitable functional group. Further, at least one of the aromatic dihydroxy compound and the aromatic dihalide compound may include a sulfone moiety. In some embodiments, the aromatic dihalide compound may include the sulfone moiety.

Exemplary aromatic dihalide compounds that may be used include 4,4′-bis(chlorophenyl)sulfone, 2,4′-bis(chlorophenyl)sulfone, 2,4-bis(chlorophenyl)sulfone, 4,4′-bis(fluorophenyl)sulfone, 2,4′-bis(fluorophenyl)sulfone, 2,4-bis(fluorophenyl)sulfone, 4,4′-bis(chlorophenyl)sulfoxide, 2,4′-bis(chlorophenyl)sulfoxide, 2,4-bis(chlorophenyl)sulfoxide, 4,4′-bis(fluorophenyl)sulfoxide, 2,4′-bis(fluorophenyl)sulfoxide, 2,4-bis(fluorophenyl)sulfoxide, 4,4′-bis(fluorophenyl)ketone, 2,4′-bis(fluorophenyl)ketone, 2,4-bis(fluorophenyl)ketone, 1,3-bis(4-fluorobenzoyl)benzene, 1,4-bis(4-fluorobenzoyl)benzene, 4,4′-bis(4-chlorophenyl)phenylphosphine oxide, 4,4′-bis(4-fluorophenyl)phenylphosphine oxide, 4,4′-bis(4-fluorophenylsulfonyl)-1,1′-biphenyl, 4,4′-bis(4-chlorophenylsulfonyl)-1,1′-biphenyl, 4,4′-bis(4-fluorophenylsulfoxide)-1,1′-biphenyl, 4,4′-bis(4-chlorophenylsulfoxide)-1,1′-biphenyl, and combinations thereof.

Non-limiting examples of suitable aromatic dihydroxy compounds that may be used include 4,4′-dihydroxyphenyl sulfone, 2,4′-dihydroxyphenyl sulfone, 4,4′-dihydroxyphenyl sulfoxide, 2,4′-dihydroxyphenyl sulfoxide, bis(3,5-dimethyl-4-hydroxyphenyl)sulfoxide, bis(3,5-dimethyl-4-hydroxyphenyl)sulfone, 4,4-(phenylphosphinyl)diphenol, 4,4′-oxydiphenol, 4,4′-thiodiphenol, 4,4′-dihydroxybenzophenone, 4,4′dihydroxyphenylmethane, hydroquinone, resorcinol, 5-cyano-1,3-dihydroxybenzene, 4-cyano-1,3,-dihydroxybenzene, 2-cyano-1,4-dihydroxybenzene, 2-methoxyhydroquinone, 2,2′-biphenol, 4,4′-biphenol, 2,2′-dimethylbiphenol 2,2′,6,6′-tetramethylbiphenol, 2,2′,3,3′,6,6′-hexamethylbiphenol, 3,3′,5,5′-tetrabromo-2,2′6,6′-tetramethylbiphenol, 4,4′-isopropylidenediphenol (bisphenol A), 4,4′-isopropylidenebis(2,6-dimethylphenol) (teramethylbisphenol A), 4,4′-isopropylidenebis(2-methylphenol), 4,4′-isopropylidenebis(2-allylphenol), 4,4′-isopropylidenebis(2-allyl-6-methylphenol), 4,4′(1,3-phenylenediisopropylidene)bisphenol (bisphenol M), 4,4′-isopropylidenebis(3-phenylphenol), 4,4′-isopropylidene-bis(2-phenylphenol), 4,4′-(1,4-phenylenediisoproylidene)bisphenol (bisphenol P), 4,4′-ethylidenediphenol (bisphenol E), 4,4′-oxydiphenol, 4,4′-thiodiphenol, 4,4′-thiobis(2,6-dimethylphenol), 4,4′-sufonyldiphenol, 4,4′-sufonylbis(2,6-dimethylphenol)4,4′-sulfinyldiphenol, 4,4′-hexafluoroisoproylidene)bisphenol (Bisphenol AF), 4,4′-hexafluoroisoproylidene) bis(2,6-dimethylphenol), 4,4′-(1-phenylethylidene)bisphenol (Bisphenol AP), 4,4′-(1-phenylethylidene)bis(2,6-dimethylphenol), bis(4-hydroxyphenyl)-2,2-dichloroethylene (Bisphenol C), bis(4-hydroxyphenyl)methane (Bisphenol-F), bis(2,6-dimethyl-4-hydroxyphenyl)methane, 2,2-bis(4-hydroxyphenyl)butane, 3,3-bis(4-hydroxyphenyl)pentane, 4,4′-(cyclopentylidene)diphenol, 4,4′-(cyclohexylidene)diphenol (Bisphenol Z), 4,4′-(cyclohexylidene)bis(2-methylphenol), 4,4′-(cyclododecylidene)diphenol, 4,4′-(bicyclo[2.2.1]heptylidene)diphenol, 4,4′-(9H-fluorene-9,9-diyl)diphenol, 3,3′-bis(4-hydroxyphenyl)isobenzofuran-1(3H)-one, 1-(4-hydroxyphenyl)-3,3′-dimethyl-2,3-dihydro-1H-inden-5-ol, 1-(4-hydroxy-3,5-dimethylphenyl)-1,3,3′,4,6-pentamethyl-2,3-dihydro-1H-in-den-5-ol, 3,3,3′,3′-tetramethyl-2,2′,3,3′-tetrahydro-1,1′-spirobi[indene]-5,6′-diol (Spirobiindane), dihydroxybenzophenone (bisphenol K), thiodiphenol (Bisphenol S), bis(4-hydroxyphenyl)diphenyl methane, bis(4-hydroxyphenoxy)-4,4′-biphenyl, 4,4′-bis(4-hydroxyphenyl)diphenyl ether, 9,9-bis(3-methyl-4-hydroxyphenyl)fluorene, N-phenyl-3,3-bis-(4-hydroxyphenyl)phthalimide, and combinations thereof.

The reaction may be effected in a polar aprotic solvent in the presence of an alkali metal compound, and optionally, in the presence of catalysts. A basic salt of an alkali metal compound may be used to effect the reaction between the dihalo and dihydroxy aromatic compounds. Exemplary compounds include alkali metal hydroxides, such as, but not limited to, lithium hydroxide, sodium hydroxide, potassium hydroxide, rubidium hydroxide, and cesium hydroxide; alkali metal carbonates, such as, but not limited to, lithium carbonate, sodium carbonate, potassium carbonate, rubidium carbonate, and cesium carbonate; and alkali metal hydrogen carbonates, such as but not limited to lithium hydrogen carbonate, sodium hydrogen carbonate, potassium hydrogen carbonate, rubidium hydrogen carbonate, and cesium hydrogen carbonate. Combinations of these compounds may also be used to effect the reaction.

Some examples of the aprotic polar solvent that may be used include N,N-dimethylformamide, N,N-diethylformamide, N,N-dimethylacetamide, N,N-diethylacetamide, N,N-dipropylacetamide, N,N-dimethylbenzamide, N-methyl-2-pyrrolidone (NMP), N-ethyl-2-pyrrolidone, N-isopropyl-2-pyrrolidone, N-isobutyl-2-pyrrolidone, N-n-propyl-2-pyrrolidone, N-n-butyl-2-pyrrolidone, N-cyclohexyl-2-pyrrolidone, N-methyl-3-methyl-2-pyrrolidone, N-ethyl-3-methyl-pyrrolidone, N-methyl-3,4,5-trimethyl-2-pyrrolidone, N-methyl-2-piperidone, N-ethyl-2-piperidone, N-isopropyl-2-piperidone, N-methyl-6-methyl-2-piperidone, N-methyl-3-ethylpiperidone, dimethylsulfoxide (DMSO), diethylsulfoxide, sulfolane, 1-methyl-1-oxosulfolane, 1-ethyl-1-oxosulfolane, 1-phenyl-1-oxosulfolane, N,N′-dimethylimidazolidinone (DMI), diphenylsulfone, and combinations thereof. The amount of solvent to be used is typically an amount that is sufficient to dissolve the dihalo and dihydroxy aromatic compounds.

The reaction may be conducted at a temperature in a range from about 100° C. to about 300° C. in some embodiments, from about 120° C. to about 200° C. in some embodiments, and from about 150° C. to about 200° C. in particular embodiments. The reaction mixture may be further dried by addition to the initial reaction mixture of, along with the polar aprotic solvent, a solvent that forms an azeotrope with water. Examples of such solvents include toluene, benzene, xylene, ethylbenzene and chlorobenzene. After removal of residual water by azeotropic drying, the reaction may be carried out at the elevated temperatures described above. The reaction is typically conducted for a time period ranging from about 1 hour to about 72 hours in some embodiments, and from about 1 hour to about 10 hours in particular embodiments.

After completion of the reaction, the copolymer may be separated from the inorganic salts, precipitated into a non-solvent and collected by filtration and drying. Examples of non-solvents include water, methanol, ethanol, propanol, butanol, acetone, methyl ethyl ketone, methyl isobutyl ketone, and combinations thereof.

The zwitterion functionalized copolymer may be then synthesized by reacting the resulting copolymer with a suitable compound (for example, sultone) at elevated temperatures.

The glass transition temperature, T_{g}, of the copolymer may be in a range from about 120° C. to about 280° C. in one embodiment, and may be in a range from about 140° C. to about 200° C. in another embodiment. The copolymer may be further characterized by the weight average molecular weight (M_{w}) obtained from gel permeation chromatography based on polystyrene standards. In one embodiment, the M_{w} of the copolymer may be in the range from about 10000 grams per mole (g/mol) to about 100000 g/mol. In another embodiment, the M_{w} may be in a range from about 10000 g/mol to about 75000 g/mol. In another embodiment, the M_{w} may be in a range from about 40000 g/mol to about 55000 g/mol.

The copolymer and the membrane including the copolymer may be further characterized by its hydrophilicity. In some embodiments, the copolymer has a contact angle with water less than about 80 degrees measured on a surface of the copolymer cast as a film on a glass substrate. In some embodiments, the copolymer has a contact angle with water less than about 50 degrees measured on a surface of the copolymer cast as a film on a glass substrate. In particular embodiments, the copolymer has a contact angle with water less than about 30 degrees measured on a surface of the copolymer cast as a film on a glass substrate.

The membrane has a hollow fiber configuration, as noted earlier. In some embodiments, a hollow-fiber membrane module including a plurality of hollow-fiber membranes is presented. A hemodialysis or hemofiltration apparatus including the hollow-fiber module is also presented.

The membranes in accordance with embodiments of the invention may be made by processes known in the art. Suitable techniques include, but are not limited to: dry-phase separation membrane formation process; wet-phase separation membrane formation process; dry-wet phase separation membrane formation process; thermally-induced phase-separation membrane formation process. Further, post membrane-formation, the membrane may be subjected to a membrane conditioning process or a treatment process prior to its use in a separation application. Representative processes may include thermal annealing to relieve stresses or pre-equilibration in a solution similar to the feed stream the membrane will contact.

In one embodiment, the membranes may be prepared by phase inversion. The phase inversion process includes 1) vapor-induced phase separation (VIPS), also called “dry casting” or “air casting”; 2) liquid-induced phase separation (LIPS), mostly referred to as “immersion casting” or “wet casting”; and 3) thermally induced phase separation (TIPS), frequently called “melt casting”. The phase inversion process can produce integrally skinned asymmetric membranes. In some embodiments, the membranes may be cross-linked to provide additional support.

The membrane may be designed and fabricated to have specific pore sizes so that solutes having sizes greater than the pore sizes may not be able to pass through. In one embodiment, the pore size may be in a range from about 0.5 nanometers to about 100 nanometers. In another embodiment, the pore size may be in a range from about 1 nanometer to about 25 nm.

A method of forming a hollow-fiber membrane is also presented. The method includes providing a casting solution comprising the copolymer as described earlier and a solvent. The method further includes extruding the casting solution through an annular channel to form the hollow-fiber membrane. Non-limiting examples of suitable solvents include N-methyl-2-pyrrolidone, dimethylacetamide, dimethylformamide, tetrahydrofuran, methyl ethyl ketone, formylpiperidine, or combinations thereof.

In some embodiments, the casting solution may further include an additive selected from the group consisting of polymers, such as, polyvinylpyrrolidone and polyethylene glycol; anti-solvents, such as, water, alcohols, glycols, glycol ethers, and salts; alkali metal halides; and combinations thereof. In some embodiments, the additive may include an alkali metal bromide, such as, but not limited to, lithium bromide, sodium bromide, potassium bromide, cesium bromide, or combinations thereof.

The additive may be present in the casting solution in an amount (total amount) in a range from about 0.1 weight percent to about 30 weight percent, in some embodiments. Further, the copolymer may be present in the casting solution in an amount in a range from about 10 weight percent to about 30 weight percent, in some embodiments.

In some embodiments, the hollow fiber membrane may include a blend of a copolymer described earlier with at least one additional polymer. The additional polymer may be blended with the copolymer to impart different properties such as better heat resistance, biocompatibility, and the like. Furthermore, the additional polymer may be added to the copolymer during the membrane formation to modify the morphology of the phase inverted membrane structure produced upon phase inversion, such as asymmetric membrane structures. In addition, at least one polymer that is blended with the copolymer may be hydrophilic or hydrophobic in nature.

In some embodiments, the copolymer is blended with a hydrophilic polymer. Non-limiting example of a suitable hydrophilic polymer includes polyvinylpyrrolidone (PVP). Non-limiting examples of other suitable hydrophilic polymers include polyoxazoline, polyethyleneglycol, polypropylene glycol, polyglycolmonoester, copolymer of polyethyleneglycol with polypropylene glycol, water-soluble cellulose derivative, polysorbate, polyethylene-polypropylene oxide copolymer, polyethyleneimine, and combinations thereof. In some embodiments, the copolymer may be further blended with polymers, such as, polysulfone, polyether sulfone, polyether urethane, polyamide, polyether-amide, polyacrylonitrile, and combinations thereof.

The membranes in accordance with some embodiments of the invention may have use in various applications, such as, bio-separation, water purification, hemofiltration, hemodialysis, ultrafiltration, nanofiltration, gas separation, microfiltration, reverse osmosis, and pervaporation. In particular embodiments, the membranes may have applications in the biopharmaceutical and biomedical field where improved hydrophilicity and biocompatibility are desired.

In some embodiments, a hollow-fiber membrane for bio-separation is presented. A hollow-fiber membrane suitable for bio-separation may be characterized in part by the protein binding. In some embodiments, the hollow-fiber membranes may have protein binding less than about 30 ng/cm^{̂2}. The membrane is composed of a copolymer in accordance with embodiments of the invention. In another aspect, the present invention relates to a bio-separation apparatus that includes a plurality of porous hollow fibers composed of the porous membranes of the present invention.

In some embodiments, the membranes in accordance with some embodiments of the invention may be used for hemodialysis. Dialysis refers to a process effected by one or more membranes in which transport is driven primarily by pressure differences across the thickness of the one or more membrane. Hemodialysis refers to a dialysis process in which biologically undesired and/or toxic solutes, such as metabolites and by-products are removed from blood. Hemodialysis membranes are porous membranes permitting the passage of low molecular weight solutes, typically less than 5,000 Daltons, such as urea, creatinine, uric acid, electrolytes and water, yet preventing the passage of higher molecular weight proteins and blood cellular elements. Hemofiltration, which more closely represents the filtration in the glomerulus of the kidney, requires even more permeable membranes allowing complete passage of solutes of molecular weight of less than 50,000 Daltons, and, in some cases, less than 20,000 Daltons

Without being bound by any theory it is believed that the copolymer in accordance with some embodiments of the present invention have the desired mechanical properties so as to support the porous hollow-fiber membrane structure during manufacture and use. In addition, the copolymer has adequate thermal properties so as not to degrade during high temperature steam sterilization processes. Further, the copolymer and the corresponding membranes have optimal biocompatibility, such that protein fouling is minimized and thrombosis of the treated blood does not occur.

### EXAMPLES

Chemicals were purchased from Aldrich and Sloss Industries and used as received, unless otherwise noted. NMR spectra were recorded on a Bruker Avance 400 (¹H, 400 MHz) spectrometer and referenced versus residual solvent shifts. Molecular weights are reported as number average (Mₙ) or weight average (M_{w}) molecular weight and were determined by gel permeation chromatography (GPC) analysis on a Perkin Elmer Series 200 instrument equipped with UV detector. Polymer thermal analysis was performed on a Perkin Elmer DSC7 equipped with a TAC7/DX thermal analyzer and processed using Pyris Software.

Glass transition temperatures were recorded on the second heating scan. Contact angle measurements were taken on a VCA 2000 (Advanced Surface Technology, Inc.) instrument using VCA optima Software for evaluation. Polymer films were obtained from casting a thin film from an appropriate solution, such as, dimethyl sulfoxide (DMSO), N-methyl-2-pyrrolidone (NMP), and dimethylacetamide (DMAC) onto a clean glass slide and evaporation of the solvent. Advancing contact angles with water (73 Dynes/cm) were determined on both sides of the film (facing air and facing glass slide). Consistently lower values were obtained on the side facing the glass slide presumably due to the smoother surface.

### Example 1

### Preparation of Polysulfone and Final Derivatization (45 Mole % Zwitterion, One Pot Synthesis, 3.25 Mole % Chain Stopper)

To a 5.0 L three neck flask immersed in an oil bath, and equipped with an overhead mechanical stirrer, shorthead distillation apparatus, and a nitrogen inlet, BPA (228.1 g, 1 mole), N-methyl piperazine diphenolamide (301.17 g, 0.8182 moles), p-cumyl phenol (12.468 g, 0.0591 moles), and 1.60 L N-methyl pyrrolidinone (NMP) were charged. This mixture was stirred at room temperature followed by addition of potassium carbonate (401.5 g, 2.909 moles) in portions, which was followed by addition of 0.8 L of toluene. This mixture was heated under a slow stream of nitrogen to remove toluene and azeotrope residual water to dry reaction the mixture. The oil bath temperature was gradually raised from 125-150° C. to remove most of the toluene (>90%). The slurry was cooled to room temperature. Difluorodiphenyl sulfone (469.63 g, 1.8482 moles) was added as a solid and the reaction temperature was gradually raised to 165° C. During the heat up a mild exotherm was observed at about 100° C. The mixture was heated and samples taken every two hours until desired molecular weight was achieved (8-10 hours).The reaction viscosity increased over the course of the run with the reaction product having an opaque greyish color. When the desired molecular weight was achieved, the reaction was diluted with 0.8 liters of NMP and cooled to 50° C. 1,3-propane sultone was added (149.7 g, 1.227 moles) and the reaction mixture gradually heated to 80° C. The reaction was complete in ˜4 hrs. Based on the solution viscosity the reaction mixture was further diluted. The mixture was precipitated into 12.0 L of water using a high speed blender, producing a white precipitate. The precipitate was collected by filtration, and re-slurried in 5.0 liters of warm water (40-50° C.) for 6 hours. The solid was collected by filtration. The resulting polymer was dried under vacuum initially at 50° C. under a purge of nitrogen for 24 hours, and an additional 24 hours at 80-100° C. under full vacuum. 950 grams of polymer was obtained after drying (95% recovery).

Similarly, copolymers having different zwitterion mole fractions were synthesized and Tables 1 and 2 provide details of the zwitterion mole fraction in the copolymers

### Example 2

### Fabrication of Hollow-Fiber Membranes

A solution containing 23 wt % zwitterion functionalized copolymer, 24 wt % polyvinylpyrrolidone, 3 wt % lithium bromide, and 50 wt % N-methylpyrrolidone was extruded through an annular nozzle into a coagulation bath containing water. The annulus bore included an inner coagulating fluid (a mixture of solvents and non-solvents) and a second, outer coagulation fluid was applied outside the annulus, forming a sheath around the freshly extruded polymer dope. The fiber was extruded through the bath at a rate of around 3-60 m/min, and was collected in a second bath for further extraction.

### Example 3

### Protein Adhesion Studies

Films cast using zwitterion-functionalized copolymers were evaluated for protein binding. Hollow fiber porous membranes were also prepared from the zwitterion-functionalized copolymers and evaluated for protein binding.

Dense films were blocked so that only the top surface (that which was exposed to air when the film was cast) was exposed to the model foulant, an HRP-labeled antibody. The surfaces were covered with a 10 μg/ml solution of HRP-Ab for 2 hours and washed thoroughly with PBS for another hour to remove loosely-adhered Ab. Using a 0.5 cm diameter biopsy punch, disks were cut from the film and 3 disks from each polymer film were transferred individually to a 24-well plate. To each well was added 0.5 ml of a solution of o-phenylene diamine, hydrogen peroxide, and citrate phosphate buffer (0.5 mg/ml, 0.015%, and 50 mM, respectively). Exactly three minutes after this solution was added, the absorbance of the solution was measured at 450 nm. The HRP enzyme on the antibody converts the o-phenylene diamine to a colored product, and thus the absorbance of the solution can be correlated to the amount of antibody that has fouled the surface of the dense film using a calibration curve. When this method is used to determine the fouling on hollow fibers, 1-inch long pieces of hollow fiber are submerged in the antibody solution for 2 hours, washed thoroughly with PBS for an additional hour, cut into quarters and the 4 quarters are transferred collectively to the wells of a 24-well plate. The enzymatic reaction and spectrophotometry are carried out as described above. Inner and outer diameter of each fiber were measured microscopically and used to calculate nominal surface area of the sample. Surface coverage was normalized by surface area.

Table 1 shows the normalized protein binding performance (normalized with respect to PSU) for commercial polysulfone (PSU) (comparative example 1) versus films cast using zwitterion-functionalized copolymers. Table 2 shows the normalized protein binding performance (normalized with respect to PSU) for commercial polysulfone (comparative example 1) versus hollow-fiber membrane formed using zwitterion-functionalized copolymers.

TABLE 1
Normalized protein binding data for films

| | Percentage mole fraction | |
|---|---|---|
| Sample | of zwitterion groups | IgG binding |
| Comparative Sample 1 | 0 | 1 |
| Sample 1 | 11 | 0.772 |
| Sample 2 | 31 | 0.587 |
| Sample 3 | 37 | 0.413 |
| Sample 4 | 50 | 0.274 |

TABLE 2
Normalized protein binding data for hollow-fiber membranes

| | Percentage mole fraction | |
|---|---|---|
| Sample | of zwitterion groups | IgG binding |
| Comparative Sample 1 | 0 | 1 |
| Sample 5 | 28 | 0.482 |
| Sample 6 | 35 | 0.417 |
| Sample 7 | 44 | 0.397 |

As illustrated in Tables 1 and 2, copolymers having zwitterion groups in an amount in a range from 30 mole percent to about 50 mole percent provided improved performance versus commercial polysulfone (PSU). The improved performance may be attributed to the presence of the zwitterion group in the copolymer.

Further, it was shown that the zwitterion group in these copolymers does not inhibit the ability of the copolymer to be made into hydrophilic hollow fiber membranes with useful porosities and mechanical performance for commercial hollow fiber applications. Lower mole fraction (e.g., 11 mole percent) of zwitterion functional groups resulted in protein adhesion performance that was lower, while higher mole fraction (e.g., 50 mole percent) of zwitterion ion functional groups although providing good protein adhesion performance, resulted in polymers that could not be fabricated into hollow-fiber membranes.

The appended claims are intended to claim the invention as broadly as it has been conceived and the examples herein presented are illustrative of selected embodiments from a manifold of all possible embodiments. Accordingly, it is the Applicants' intention that the appended claims are not to be limited by the choice of examples utilized to illustrate features of the present invention. As used in the claims, the word “comprises” and its grammatical variants logically also subtend and include phrases of varying and differing extent such as for example, but not limited thereto, “consisting essentially of” and “consisting of.” Where necessary, ranges have been supplied; those ranges are inclusive of all sub-ranges there between. It is to be expected that variations in these ranges will suggest themselves to a practitioner having ordinary skill in the art and where not already dedicated to the public, those variations should where possible be construed to be covered by the appended claims. It is also anticipated that advances in science and technology will make equivalents and substitutions possible that are not now contemplated by reason of the imprecision of language and these variations should also be construed where possible to be covered by the appended claims.

1. A hollow-fiber membrane, comprising:
a copolymer comprising sulfone structural units having a formula (I) and zwitterion-functionalized structural units having a formula (II): 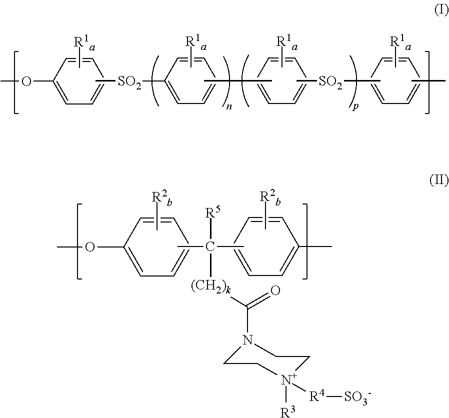
wherein “a” and “b” are independently at each occurrence 0, 1, 2, 3, or 4;
“n” and “p” are independently 0 or 1;
“k” is in a range from 0 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical;
R³ and R⁵ are independently a hydrogen atom, a C₁-C₁₂ aliphatic radical, a C₃-C₁₂ cycloaliphatic radical, or a C₃-C₁₂ aromatic radical;
R⁴ is a bond, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

2. The hollow-fiber membrane of claim 1, wherein the mole fraction of the zwitterion-functionalized structural units in the copolymer is in a range from about 30 mole percent to about 50 mole percent.

3. The hollow-fiber membrane of claim 1, wherein the sulfone structural units have a formula (III): 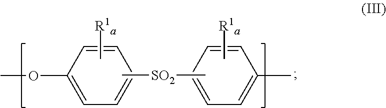
wherein “a” is independently at each occurrence 0, 1, 2, 3, or 4; and
R¹ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

4. The hollow-fiber membrane of claim 1, wherein the zwitterion-functionalized structural structural units have a formula (IV): 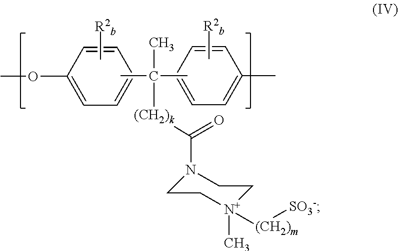
wherein “b” is independently at each occurrence 0, 1, 2, 3, or 4;
“k” is a number in a range from 0 to 10;
“m” is a number in a range from 1 to 10;and
R² is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

5. The hollow-fiber membrane of claim 1, wherein the copolymer further comprises structural units having a formula (V): 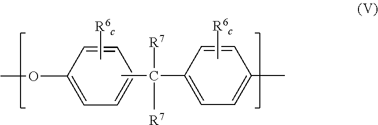
wherein “c” is independently at each occurrence 0, 1, 2, 3, or 4;
R⁶ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
R⁷ is independently at each occurrence a C₁-C₁₂ aliphatic radical, a C₃-C₁₂ cycloaliphatic radical, or a C₃-C₁₂ aromatic radical.

6. The hollow-fiber membrane of claim 1, wherein the molecular weight of the copolymer is in a range from about 10000 g/mol to about 75000 g/mol.

7. A hollow-fiber module comprising a plurality of hollow-fiber membranes of claim 1.

8. A hemodialysis or hemofiltration apparatus comprising the hollow-fiber module of claim 7.

9. A hollow-fiber membrane, comprising a copolymer comprising sulfone structural units having a formula (III) and zwitterion-functionalized structural units having a formula (IV): 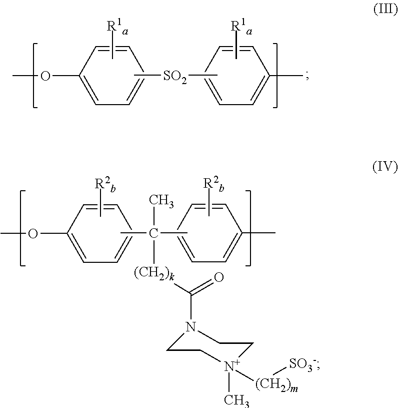
wherein “a” and “b” are independently at each occurrence 0, 1, 2, 3, or 4;
“k” is a number in a range from 0 to 10;
“m” is a number in a range from 1 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent.

10. The hollow-fiber membrane of claim 9, wherein the mole fraction of the zwitterion-functionalized structural units in the copolymer is in a range from about 30 mole percent to about 50 mole percent.

11. A hemodialysis or hemofiltration apparatus comprising a hollow-fiber module comprising a plurality of hollow-fiber membranes of claim 9.

12. A method of forming a hollow-fiber membrane, comprising:
(A) providing a casting solution comprising a copolymer and a solvent, wherein the copolymer comprises sulfone structural units having a formula (I) and zwitterion-functionalized structural units having a formula (II): 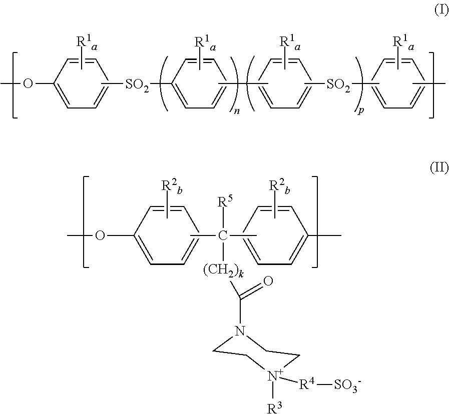
wherein “a” and “b” are independently at each occurrence 0, 1, 2, 3, or 4;
“n” and “p” are independently 0 or 1;
“k” is in a range from 0 to 10;
R¹ and R² are independently at each occurrence a hydrogen atom, a halogen atom, a nitro group, a C₁-C₁₂ aliphatic radical, a C₃-C₁₂ cycloaliphatic radical, or a C₃-C₁₂ aromatic radical;
R³ and R⁵ are independently a hydrogen atom, a C₁-C₁₂ aliphatic radical, a C₃-C₁₂ cycloaliphatic radical, or a C₃-C₁₂ aromatic radical;
R⁴ is a bond, a C₁-C₁₂ aliphatic radical, a C₃-C₁₂ cycloaliphatic radical, or a C₃-C₁₂ aromatic radical; and
wherein a mole fraction of the zwitterion-functionalized structural units in the copolymer is less than about 50 mole percent; and
(B) extruding the casting solution through an annular channel to form the hollow-fiber membrane.

13. The method of claim 12, wherein the mole fraction of the zwitterion-functionalized structural units in the copolymer is in a range from about 30 mole percent to about 50 mole percent.

14. The method of claim 12, wherein the sulfone structural units have a formula (III): 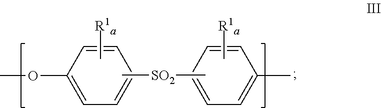
wherein “a” is independently at each occurrence 0, 1, 2, 3, or 4; and
R¹ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

15. The method of claim 12, wherein the zwitterion-functionalized structural structural units have a formula (IV): 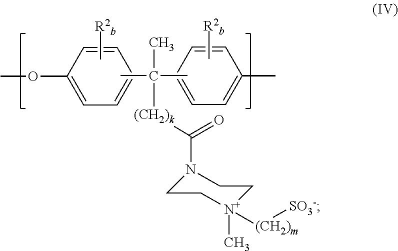
wherein “b” is independently at each occurrence 0, 1, 2, 3, or 4;
“k” is a number in a range from 0 to 10;
“m” is a number in a range from 1 to 10; and
R² is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical.

16. The method of claim 12, wherein the copolymer further comprises structural units having a formula (V): 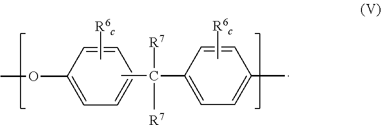
wherein “c” is independently at each occurrence 0, 1, 2, 3, or 4;
R⁶ is independently at each occurrence a hydrogen atom, a halogen atom, a nitro group, a C₁-C₁₂ aliphatic radical, a C₃-C₁₂ cycloaliphatic radical, or a C₃-C₁₂ aromatic radical; and
R⁷ is independently at each occurrence a C₁-C₁₂ aliphatic radical, a C₃-C₁₂ cycloaliphatic radical, or a C₃-C₁₂ aromatic radical.

17. The method of claim 12, wherein the casting solution further comprises an additive selected from the group consisting of polymers, anti-solvents, alkali metal halides, and combinations thereof.

18. The method of claim 12, wherein the casting solution further comprises alkali metal bromide.

19. The method of claim 12, wherein the additive is present in the casting solution in an amount in a range from about 0.1 weight percent to about 30 weight percent.

20. The method of claim 12, wherein the copolymer is present in the casting solution in an amount in a range from about 10 weight percent to about 30 weight percent.
